(19) United States
(12) Patent Application Publication
(10) Pub. No.: US 2018/0338746 A1
(43) Pub. Date: Nov. 29, 2018

(54) POWER MANAGEMENT METHOD AND ULTRASOUND APPARATUS THEREOF

(71) Applicant: Leltek Inc., New Taipei City (TW)

(72) Inventors: Chien-Hsin Wu, New Taipei City (TW); Ying-Yi Liu, Taipei City (TW); Sheng-Chang Peng, Taipei City (TW); Kuo-Ping Liu, Taipei City (TW)

(73) Assignee: Leltek Inc., New Taipei City (TW)

(21) Appl. No.: 15/603,462

(22) Filed: May 24, 2017

(51) Int. Cl. A61B 8/00; A61B 8/08; A61B 8/14

(52) U.S. Cl. CPC A61B 8/56; A61B 8/4254; A61B 8/14; A61B 8/5207; A61B 8/4444

(57) ABSTRACT

The disclosure is directed to a power management method and an ultrasound apparatus using the same. The ultrasound apparatus includes an ultrasound probe, an TRX circuit, a position sensor, and a processor. In the disclosure, power is supplied to the ultrasound apparatus, and an ultrasound scanning is performed. During the ultrasound scanning, position information of the ultrasound probe is being continuously detected by the position sensor. Whether the ultrasound probe is in a static state during the ultrasound scanning is determined. When determined that the ultrasound probe is in the static state during the ultrasound scanning, a power saving mode is entered, such that the ultrasound scanning is stopped. Within the power saving mode, the position information is continuously detected. Whether the ultrasound probe is still in the static state under the power saving mode is determined. When determined that the ultrasound probe is still in the static state under the power saving mode, no power is being supplied to the TRX circuit.

### BACKGROUND

### Technical Field

The disclosure relates to a power management method and an ultrasound apparatus thereof, where the power management method is adapted to the ultrasound apparatus.

### Description of Related Art

Ultrasound has been widely used in medical diagnosis, military radar system and rusting detection. In the medical diagnosis system, the ultrasound is often used in imaging internal body structures such as tendons, muscles, joints, vessels and internal organs. Since the image obtained by the ultrasound may clearly show the internal body, illness or disease may be correctly examined, and appropriate treatment could be chosen and performed by medical personnel, so as to effectively cure a patient.

Most conventional ultrasound devices are manually activated and deactivated. That is to say, those ultrasound devices will not be shut down automatically. However, for maintaining the operation of the ultrasound device, power consumption is considerable, which usually leads to the result that the portable type ultrasound device can only be used for a short time. Further, heat generated from the continually operated ultrasound device also cause the examinee feeling uncomfortable. Therefore, it is expected that the ultrasound device may be temporarily shut down or turned into a low power consumption mode under automatic control. In other words, it is still a goal of effort for those technicians of the field to provide an efficient and accurate power management mechanism for ultrasound devices.

### SUMMARY

The disclosure is directed to a power management method and an ultrasound apparatus thereof, by which power of the ultrasound apparatus is efficiently and accurately managed.

An embodiment of the disclosure provides a power management method, adapted to an ultrasound apparatus including an ultrasound probe, a TRX (transmit/receive) circuit (which includes an analog-front-end (AFE) circuit and a pulser), a position sensor, a power source and a processor (such as FPGA, MCU, . . . etc.) The power management method includes following steps. Power is supplied to the TRX circuit and the processor. An ultrasound scanning is performed by the TRX circuit, the processor is capturing and calculating ultrasound images. During the ultrasound scanning, position information of the ultrasound apparatus is continuously detected. According to the detected position information, when determined that the ultrasound probe is in a static state during the ultrasound scanning, the ultrasound apparatus enters a power saving mode. In the power saving mode, the ultrasound scanning is stopped and the processor is suspended. Under the power saving mode, the position information of the ultrasound probe is periodically checked by the processor. When determined that the ultrasound probe is still in the static state under the power saving mode, according to the detected position information, the processor controls the power supply to stop supply power to the TRX circuit.

In an embodiment of the present disclosure, the ultrasound probe is determined as in the static state when the movement of the ultrasound probe, recognized from the detected position information, falls in a predefined range in a period.

In an embodiment of the present disclosure, when determined that the ultrasound probe is not in the static state during the ultrasound scanning, the ultrasound scanning continuously proceeds, and the position information of the ultrasound probe is continuously detected.

In an embodiment of the present disclosure, when determined that the ultrasound probe is in the static state during the ultrasound scanning, the power management method goes through following steps before entering the power saving mode. A freeze mode is entered to stop the ultrasound scanning performed by the TRX circuit and the processor, wherein the latest ultrasound image is continuously displayed. Within the freeze mode, the position information of the ultrasound probe is continuously detected. If the ultrasound probe keeps in the static state under the freeze mode for a predefined period, the power saving mode is entered to suspend the processor.

In an embodiment of the present disclosure, when determined that the ultrasound probe is not in the static state under the freeze mode, the ultrasound scanning performed by the TRX circuit, the ultrasound probe and the processor is restarted, and the position information of the ultrasound probe is continuously detected afterward.

In an embodiment of the present disclosure, when determined that the ultrasound probe is not in the static state under the power saving mode, the processor is woke up from the suspension, and the ultrasound scanning is restarted. The position information of the ultrasound probe is continuously detected afterward.

The disclosure also includes an embodiment of an ultrasound apparatus performing a power management method. The ultrasound apparatus includes an ultrasound probe, a TRX circuit (includes AFE and pulser circuits), a position sensor, a power source, and a processor. The position sensor is disposed close to the ultrasound probe. The power source is connected to the TRX circuit, the sensor, and the processor. The processor controls the power source to supply power to the TRX circuit, and controls the TRX circuit to perform the ultrasound scanning. During the ultrasound scanning, the position sensor continuously detects the position information of the ultrasound probe, and the processor determines whether the ultrasound probe is in a static state accordingly. When determined that the ultrasound probe is in the static state during the ultrasound scanning, the processor controls the ultrasound apparatus to enter the freeze mode, such that the ultrasound scanning is stopped. When freeze mode, the sensor continuously detects the position information of the ultrasound probe, and the processor determines whether the ultrasound probe is still in the static state. If the processor determines the ultrasound probe is still in the static state under the freeze mode, the apparatus enters the power saving mode and the processor controls the power source to stop supplying the power to the TRX circuit.

According to the description above, the power management method detects position information of the ultrasound probe to determine whether the ultrasound probe is being operated or in the static state. When the ultrasound probe is in the static state, the ultrasound apparatus is controlled to enter the power saving mode and the power is stopped being supplied to the TRX circuit. By such, the power of the ultrasound apparatus is efficiently and accurately managed.

In order to make the aforementioned and other features and advantages of the disclosure comprehensible, several exemplary embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram illustrating an ultrasound apparatus according to an embodiment of the present disclosure.

FIG. 2 is a flowchart illustrating a power management method according to an embodiment of the present disclosure.

FIG. 3 is a flowchart illustrating a power management method according to another embodiment of the present disclosure.

### DESCRIPTION OF EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

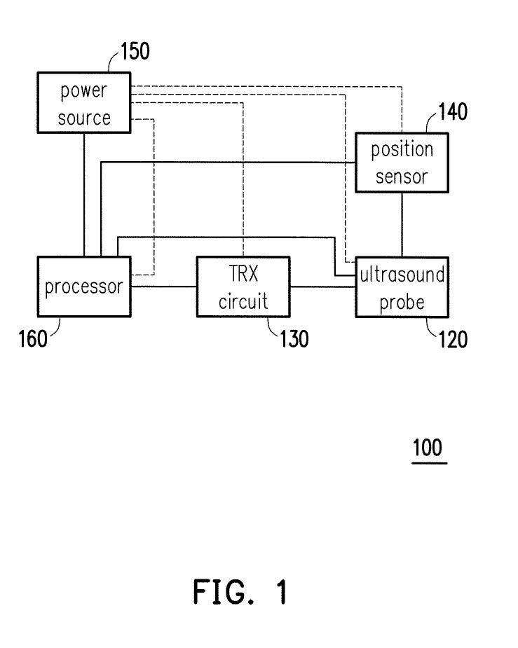

FIG. 1 is a schematic diagram illustrating an ultrasound apparatus according to an embodiment of the present disclosure. Referring to FIG. 1, in the present embodiment of the present disclosure, the ultrasound apparatus 100 includes an ultrasound probe 120, a transmit-and-receive (TRX) circuit 130 (includes the analog-front-end (AFE) and pulser), a position sensor 140, a power source 150 and a processor 160. In an embodiment of the disclosure, the ultrasound apparatus 100 may be implemented in an electronic apparatus with computation ability, such as a laptop computer, a tabular computer, a smart device and a mobile phone, but it is not limited herein. With the ultrasound apparatus 100, ultrasound scanning may be performed, such that a plurality of ultrasound images are captured and displayed.

In an embodiment of the present disclosure, the ultrasound probe 120 is applied for emitting ultrasound waves and receiving echoes of the ultrasound waves. The ultrasound probe 120 may include a plurality of transducer elements which are horizontally arranged in a line.

In an embodiment of the present disclosure, the TRX circuit 130 is connected to the ultrasound probe 120 and communicates with the ultrasound probe 120 in the ultrasound apparatus 100. In other words, the TRX circuit 130 performs the transmitting and receiving ultrasound signal by accessing the ultrasound probe 120, controlled by the processor 160. The TRX circuit 130 may be composed of multiple channels of analog-front-end (AFE) circuits and pulsers, multiple filters and multiple amplifiers, but it is not limited herein.

In an embodiment of the present disclosure, the position sensor 140 disposed on the ultrasound probe 120 may be, for example, an acceleration sensor, a gravity sensor (G sensor), a gyroscope, a digital compass, an electronic compass, or similar sensors, or a combination of the above sensors for detecting position information which may include acceleration, orientation, inclination angle, rotation angle and facing direction of the ultrasound probe 120. Based on the position information from the position sensor 140, movement of the ultrasound probe 120 may be deduced by the processor 160.

In an embodiment of the present disclosure, the power source 150 in the ultrasound apparatus 100 may be, for example, battery or power supply unit (PSU), but it is not limited herein. The power source 150 is connected to the ultrasound probe 120, the TRX circuit 130, the position sensor 140 and the processor 160 for power supplement.

In an embodiment of the present disclosure, the processor 160 may include one or more of a field programmable gate array (FPGA), a programmable logic device (PLD), an application specific integrated circuit (ASIC), a central processing unit (CPU), a programmable general purpose or special purpose microprocessor, a micro-controller unit (MCU), or other similar devices or a combination thereof.

It should be noted that, in addition to the ultrasound probe 120, the TRX circuit 130, the position sensor 140, the power source 150 and the processor 160 mentioned above, the ultrasound apparatus 100 may further include storage device, power button, display device, and user interface, but it is not limited herein. Furthermore, the ultrasound apparatus 100 may also include an input/output circuit (not shown) that provides ultrasound data captured by the ultrasound probe to a display, which may be external on a mobile phone, notebook, etc. The display may display the captured ultrasound images according to the ultrasound data.

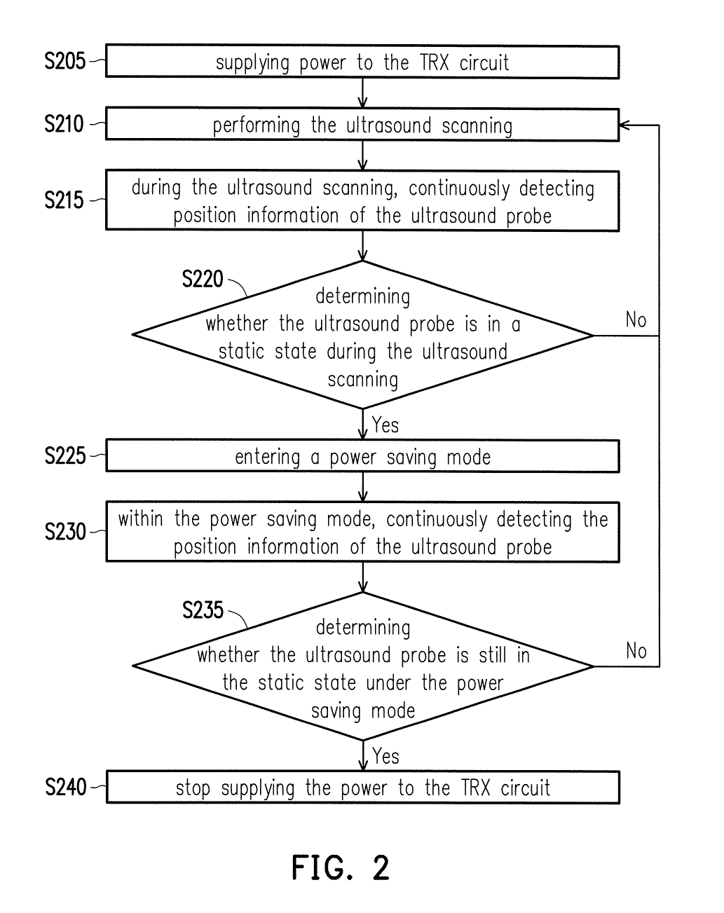

FIG. 2 is a flowchart illustrating a power management method according to an embodiment of the present disclosure. In the present embodiment, the power management method is, for example, adapted to the ultrasound apparatus 100 shown in FIG. 1, but it is not limited herein. The power management method of the present embodiment is described below with reference of various components of the ultrasound apparatus 100.

Referring to FIG. 1 and FIG. 2, when the ultrasound apparatus 100 is activated, power is supplied to the components of the ultrasound apparatus 100. Especially, under the control of the processor 160, power is supplied to the TRX circuit 130 (step S205). The ultrasound apparatus 100 may be activated through pressing the power button of the ultrasound apparatus 100, but it is not limited herein. Further, the ultrasound scanning is performed (step S210) by the ultrasound apparatus 100.

To be more specific, the ultrasound probe 120 and the TRX circuit 130 are controlled by the processor 160 for performing the ultrasound scanning to capture ultrasound images. The processor 160 controls the TRX circuit 130 and the ultrasound probe 120 to emit ultrasound waves and to detect echoes that are produced when the emitted ultrasound waves are reflected by objects. Further, according to the detected echoes, multiple ultrasound images are generated by the processor 160's calculation.

During the ultrasound scanning, the position sensor 140 disposed on the ultrasound probe 120 continuously detects position info illation of the ultrasound probe 120 (step S215). The position information which may include at least one of acceleration, orientation, inclination angle, rotation angle and facing direction of the ultrasound probe 120 is sent to the processor 160, and the processor 160 determines whether the ultrasound probe 120 is in a static state during the ultrasound scanning (step S220) according to the detected position information.

In an embodiment of the present disclosure, the ultrasound probe 120 is determined to be in the static state when the processor 160 determines that movement of the ultrasound probe 120 (within a period) falls in a predefined range according to the detected position information. On the other hand, the ultrasound probe 120 is determined to be not in the static state when the processor 160 determines that the movement of the ultrasound probe 120 is greater than a predefined range. The period may be few seconds, a half minute or one minute, but it is not limited herein. However, in another embodiment of the present disclosure, the ultrasound probe 120 is determined as in the static state by the processor 160 only when the ultrasound probe 120 is held still for the period.

Referring to FIG. 1 and FIG. 2, when determined that the ultrasound probe 120 is in the static state during the ultrasound scanning, the processor 160 controls the ultrasound apparatus 100 to enter a power saving mode (step S225), such that the ultrasound scanning is stopped. In other words, since the ultrasound probe 120 has not been moved for a period of time, the processor 160 temporarily suspends the ultrasound scanning. Specifically, in the power saving mode, the operations of the TRX circuit 130 and the ultrasound probe 120 are suspended.

By contrast, when determined that the ultrasound probe 120 is not in the static state during the ultrasound scanning, the processor 160 controls the the TRX circuit 130 and the ultrasound probe 120 to maintain the ultrasound scanning (step S210), and the sensor 140 continuously detects the position information of the ultrasound probe 120 (step S215).

Referring to FIG. 1 and FIG. 2, within the power saving mode, the sensor 140 continuously detects the position information of the ultrasound probe 120 (step S230), and the processor 160 determines whether the ultrasound probe 120 is still in the static state under the power saving mode (step S235) according to the detected position information. When determined that the ultrasound probe 120 is still in the static state under the power saving mode, the processor 160 may recognize that the whole scanning process is finished, and controls the power source 150 to stop supplying the power to the TRX circuit 130 (step S240). Also, the processor 160 may control the power source 150 to stop supplying power to other components in the ultrasound apparatus 100. By contrast, when determined that the ultrasound probe 120 is not in the static state under the power saving mode, the processor 160 restarts the ultrasound scanning (step S210), and the position sensor 140 continuously detects the position information of the ultrasound probe (step S215) during the ultrasound scanning.

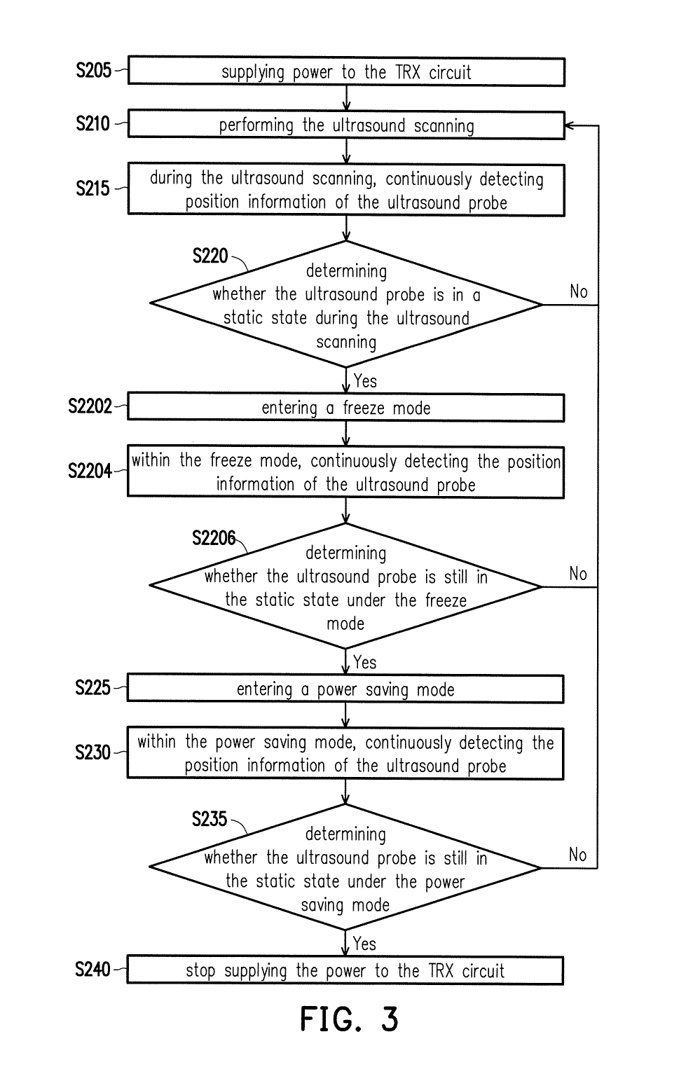

FIG. 3 is a flowchart illustrating a power management method according to another embodiment of the present disclosure. In the present embodiment, the power management method is also, for example, adapted to the ultrasound apparatus 100 shown in FIG. 1, but it is not limited herein. The difference between the power management methods shown in FIG. 2 and FIG. 3 is that, the power management method shown in FIG. 3 (step S2202) further includes a freeze mode.

To be more specific, when determined that the ultrasound probe is in the static state during the ultrasound scanning, before entering the power saving mode (step S225), the processor 160 controls the ultrasound apparatus 100 to enter a freeze mode (step S2202) to stop the ultrasound scanning performed by the TRX circuit 130 and the ultrasound probe 120, while the latest ultrasound image is fixed and continuously displayed. Here, the processor 160 may first consider that the ultrasound probe 120 is temporarily stopped for scanning the same place, so even the ultrasound scanning is suspended, the latest ultrasound image captured by the ultrasound probe 120 will still be displayed for a while. In other words, the latest ultrasound image may still be transmitted (or output) to a display.

In addition, within the freeze mode, the sensor 140 continuously detects the position information of the ultrasound probe 120 (step S2204), and the processor 160 determines whether the ultrasound probe 120 is still in the static state under the freeze mode (step S2206) according to the detected position information. When determined that the ultrasound probe 120 is still in the static state under the freeze mode, the processor 160 controls the ultrasound apparatus 100 to enter the power saving mode (step S225) to turn off the power source to supply power to the TRX circuit and/or ultrasound probe. In contrast, when determined that the ultrasound probe 120 is not in the static state under the freeze mode, the processor 160 controls the TRX circuit 130 and the ultrasound probe 120 to restart the ultrasound scanning (step S210), and the sensor 140 continuously detects the position information of the ultrasound probe 120 (step S215) during the ultrasound scanning.

Other steps of the power management method shown in FIG. 3 could be referred from the power management method shown in FIG. 2, so it is not repeated herein.

In summary, in the power management method and the ultrasound apparatus, through the detected position information of the ultrasound probe, whether the ultrasound probe is in the static state is determined. When the ultrasound probe is in the static state, the ultrasound apparatus is controlled to enter the power saving mode or the power is stopped being supply to the TRX circuit. By such, power of the ultrasound apparatus is efficiently and accurately managed.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A power management method, adapted to an ultrasound apparatus which has an ultrasound probe, a transmit-and-receive (TRX) circuit and a processor, the power management method comprising:
supplying power to the TRX circuit;
performing an ultrasound scanning by the TRX circuit, the ultrasound probe and the processor for capturing, calculating, and generating ultrasound images;
during the ultrasound scanning, continuously detecting position information of the ultrasound probe;
determining whether the ultrasound probe is in a static state during the ultrasound scanning according to the detected position information;
when determined that the ultrasound probe is in the static state during the ultrasound scanning, entering a power saving mode to stop ultrasound scanning and suspend the processor;
within the power saving mode, continuously detecting the position information of the ultrasound probe;
determining whether the ultrasound probe is still in the static state under the power saving mode according to the detected position information; and
when determined that the ultrasound probe is still in the static state under the power saving mode, stop supplying the power to the TRX circuit.

2. The power management method according to claim 1, wherein the ultrasound probe is determined as in the static state when movement of the ultrasound probe within a period recognized from the detected position information falls in a predefined range.

3. The power management method according to claim 1, further comprising:
when determined that the ultrasound probe is not in the static state during the ultrasound scanning, continuously performing the ultrasound scanning and continuously detecting the position information of the ultrasound probe.

4. The power management method according to claim 1, wherein when determined that the ultrasound probe is in the static state during the ultrasound scanning, before entering the power saving mode, the power management method comprises:
entering a freeze mode to stop the ultrasound scanning performed by the TRX circuit and the ultrasound probe, wherein the latest ultrasound image is fixed and continuously displayed;
within the freeze mode, continuously detecting the position information of the ultrasound probe;
determining whether the ultrasound probe is still in the static state under the freeze mode according to the detected position information; and
when determined that the ultrasound probe is still in the static state under the freeze mode, entering the power saving mode to suspend the processor.

5. The power management method according to claim 4, wherein when determined that the ultrasound probe is not in the static state under the freeze mode, restarting the ultrasound scanning performed by the TRX circuit and the ultrasound probe, and continuously detecting the position information of the ultrasound probe.

6. The power management method according to claim 1, further comprising:
when determined that the ultrasound probe is not in the static state under the power saving mode, restarting the ultrasound scanning performed by the TRX circuit and the ultrasound probe, and continuously detecting the position information of the ultrasound probe.

7. An ultrasound apparatus, comprising:
an ultrasound probe;
an transmit-and-receive (TRX) circuit;
a position sensor, disposed on the ultrasound probe;
a power source connected to the ultrasound probe, the TRX circuit and the sensor; and
a processor, connected to the power source, controlling the power source to supply power to the TRX circuit, and controlling the TRX circuit and the ultrasound probe to perform an ultrasound scanning for capturing ultrasound images,
during the ultrasound scanning, the sensor continuously detects position information of the ultrasound probe, and the processor determines whether the ultrasound probe is in a static state during the ultrasound scanning according to the detected position information,
when determined that the ultrasound probe is in the static state during the ultrasound scanning, the processor controls the ultrasound apparatus to enter a power saving mode, such that the ultrasound scanning is stopped,
within the power saving mode, the sensor continuously detects the position information of the ultrasound probe, and the processor determines whether the ultrasound probe is still in the static state under the power saving mode according to the detected position information,
when determined that the ultrasound probe is still in the static state under the power saving mode, the processor controls the power source to stop supplying the power to the TRX circuit.

8. The ultrasound apparatus according to claim 7, wherein the ultrasound probe is determined as in the static state by the processor when movement of the ultrasound probe within a period recognized from the detected position information falls in a predefined range.

9. The ultrasound apparatus according to claim 7, wherein when determined that the ultrasound probe is not in the static state during the ultrasound scanning, the processor controls the TRX circuit, and the ultrasound probe to continuously perform the ultrasound scanning, and the sensor continuously detects the position information of the ultrasound probe.

10. The ultrasound apparatus according to claim 7, wherein when determined that the ultrasound probe is in the static state during the ultrasound scanning, before entering the power saving mode, the processor controls the ultrasound apparatus to enter a freeze mode to stop the ultrasound scanning performed by the TRX circuit and the ultrasound probe, and the latest ultrasound image is continuously displayed,
within the freeze mode, the sensor continuously detects the position information of the ultrasound probe, and the processor determines whether the ultrasound probe is still in the static state under the freeze mode according to the detected position information,
when determined that the ultrasound probe is still in the static state under the freeze mode, the processor controls the ultrasound apparatus to enter the power saving mode.

11. The ultrasound apparatus according to claim 10, wherein when determined that the ultrasound probe is not in the static state under the freeze mode, the processor controls the TRX circuit and the ultrasound probe to restart the ultrasound scanning, and the sensor continuously detects the position information of the ultrasound probe.

12. The ultrasound apparatus according to claim 7, wherein when determined that the ultrasound probe is not in the static state under the power saving mode, the processor restarts the ultrasound scanning, and the sensor continuously detects the position information of the ultrasound probe.
